(19) United States
(12) Patent Application Publication
(10) Pub. No.: US 2006/0009486 A1
(43) Pub. Date: Jan. 12, 2006

(54) Composition and method for treatment and prevention of coronary artery disease

(75) Inventor: Odes Mitchell, Arlington, TX (US)

Correspondence Address: DECKER, JONES, MCMACKIN, MCCLANE, HALL &;BATES, P.C., BURNETT PLAZA 2000, 801 CHERRY STREET, UNIT #46, FORT WORTH, TX, 76102-6836, US

(73) Assignee: GM Pharmaceuticals Inc.

(21) Appl. No.: 10/886,389

(22) Filed: Jul. 7, 2004

(51) Int. Cl. A61K 31/47

(52) U.S. Cl. 514/310

(57) ABSTRACT

A composition and method for lowering total cholesterol levels and triglyceride levels and for reducing homocysteine levels in a human has policosanol, which is a mixture of fatty alcohols derived from sugar cane wax, omega fatty acids which are DHA (docosahexaenoic acid) and EPA (eicosapentaenoic acid) and vitamins B6 (pyridoxine hydrochloride), B12 (cobalamin) and folic acid.

### FIELD OF THE INVENTION

The present invention relates to compositions and methods for lowering total cholesterol levels, lowering triglyceride levels, reducing homocysteine levels in humans and raising HDL cholesterol levels.

### BACKGROUND OF THE INVENTION

Elevated total serum cholesterol levels (greater than 200 mg/dl) have been indicated as a major risk factor for heart disease, which is the leading cause of death among Americans. However the risk factor is not only related to an elevated total cholesterol. More recent research has examined the individual components of the lipid profile in addition to the total cholesterol. Elevated LDL (low density lipoproteins) (greater than 130 mg/dl) is a major risk factor, as these loosely packed lipoproteins are more likely to lodge in the cardiovascular system, leading to the formation of plaque. Low levels (less than 40 mg/dl) of HDL (high density lipoproteins), are also risk factors, as these lipoproteins work to sweep from the bloodstream the cholesterol that is clogging the arteries. Also, an elevated cholesterol ratio (greater than 5 mg/dl) increases the risk for developing heart disease.

Homocysteine, an amino acid, and a by-product created in the digestion of proteins, has been linked to cardiovascular disease. It is a risk factor for heart attack and stroke. Studies show homocysteine damages the smooth vascular wall tissue, creating a scratch in the inside of the vessel where plaque can build up. Homocysteine causes toxic superoxide radicals to form in the blood, which in turn kill cells in the blood vessel walls. Once damaged, the affected area swells, and forms a rough spot where sticky cholesterol start to collect to form plaque. The problem is further exacerbated because homocysteine travels around the blood stream linked to low density lipoproteins (LDL's). As homocysteine levels increase, individuals are at a higher risk for the formation of blood clots, which can lead to a heart attack or stroke.

Statin drugs are used to regulate cholesterol levels in individuals. Statin drugs are expensive, are ineffective in some individuals and have undesirable side effects such as liver damage and depletion of the body's natural production of Co enzyme Q10, which would predispose an individual to heart disease, the very condition that statin drugs are supposed to control.

### SUMMARY OF THE INVENTION

It is an object of the present invention to provide a composition for ingestion by humans to regulate cholesterol and triglyceride levels and to reduce homocysteine levels.

It is another object of the present invention to provide a method for administering a composition to regulate cholesterol and triglyceride levels as well as reduce homocysteine levels.

The present invention provides an oil formulation for regulating total cholesterol and triglyceride levels and for reducing homocysteine levels in a human comprising effective amounts of policosanol, docosahexaenoic acid, eicosapentaenoic acid, vitamin B6, vitamin B12 and folic acid.

In accordance with one aspect of the present invention, the oil formulation contains the following amounts: policosanol—2 mg-60 mg, DHA—20 mg-1000 mg, EPA—20 mg-1000 mg, vitamin B6—2 mg-50 mg, vitamin B12—25 mcg-2 mg and folic acid—0.01 mg-5 mg.

In accordance with one aspect of the present invention, the oil formulation contains the following amounts: policosanol—20 mg, DHA—400 mg, EPA—400 mg, vitamin B6—25 mg, vitamin B12—400 mcg and folic acid—1 mg.

The present invention also provides a method for regulating total cholesterol and triglyceride levels and for reducing homocysteine levels in a human, comprising periodically administering orally a formulation having of policosanol, docosahexaenoic acid, eicosapentaenoic acid, vitamin B6, vitamin B12 and folic acid.

The present invention also provides a method for regulating total cholesterol and triglyceride levels and for reducing homocysteine levels in a human, comprising periodically administering orally a formulation having policosanol—2 mg-60 mg, docosahexaenoic acid—20 mg-1000 mg, eicosapentaenoic acid—20 mg-1000 mg, vitamin B6—2 mg-50 mg, vitamin B12—25 mcg-2 mg and folic acid—0.01 mg-5 mg.

The present invention also provides a method for regulating total cholesterol and triglyceride levels and for reducing homocysteine levels in a human, comprising periodically administering orally a formulation having policosanol—20 mg, docosahexaenoic acid—400 mg, eicosapentaenoic acid—400 mg, vitamin B6—25 mg, vitamin B12—400 mcg and folic acid—1 mg.

### DETAILED DESCRIPTION OF THE INVENTION

The present invention involves a composition for oral ingestion that contains policosanol, DHA (docosahexaenoic acid), EPA (eicosapentaenoic acid), vitamin B6 (pyridoxine hydrochloride), vitamin B12 (Cobalamin), folic acid, as well as other healthy filler ingredients. More specifically, this formulated product lowers total cholesterol levels, cholesterol ratio levels, LDL cholesterol levels, triglycerides, reduces homocysteine levels, reduces tendency for thrombosis, reduces sudden death from ventricular fibrillation, and raises HDL cholesterol levels in human individuals.

Policosanol is a mixture of fatty alcohols derived from wax of sugar cane. Policosanol is a lineal saturated primary alcohol containing 20 or more carbon atoms.

The amount of policosanol is 2 mg-60 mg. Policosanol produces a significant reduction in serum total cholesterol, and the harmful LDL cholesterol levels. Triglycerides are also significantly reduced. The protective HDL values are increased, and improvements made in the ratio of total cholesterol. Policosanol lowers total cholesterol and lowers LDL cholesterol by inhibiting cholesterol manufacture, inserting no direct inhibition on HMG-COA reductase. It increases LDL receptor dependant processing by increasing the binding of LDL to its receptor, improving the transport of LDL into the liver, and enhancing the breakdown of LDL cholesterol. Policosanol also reduces the cholesterol content in different tissues such as liver, heart and fatty tissue. These cholesterol lowering effects with policosanol are persistent, as policosanol will not lose effect over time. Policosanol reduces platelet aggregation by altering prostaglandin synthesis, without affecting coagulation. Policosanol prevents and reverses atheroscerotic lesions and thrombosis. Policosanol prevents intimal thickening and smooth muscle proliferation. Policosanol is an effective antioxidant in preventing LDL oxidation.

These improvements in lipid profiles compare favorably to results observed with statin drugs. But while statin drugs have well known side effects, policosanol is considered completely safe. Policosanol has not been shown to produce any adverse drug interaction. It can be used for diabetics, elderly, and even patients with impaired liver function, or severe liver damage without fear of side effects.

DHA (docosahexaenoic acid) and EPA (eicosapentaenoic acid) are long chain omega-3 fatty acids that have been shown to improve heart function, and reduce the risk of heart disease. One of the most important findings is of a reduction in sudden death from ventricular fibrillation. Omega-3 fatty acids also reduce the tendency for thrombosis (formation of blood clots) and thus help prevent myocardial infarction. EPA and DHA also have several actions that inhibit the development of atherosclerosis. They have a substantial triglyceride-lowering effect, and also raised levels of HDL (good cholesterol).

Long chain omega-3 fatty acids produce an anti-inflammatory action throughout the body, reducing arterial inflammation, a condition associated with cardiac death. The amount of DHA and EPA is 20 mg-1000 mg each.

Vitamin B6 (pyridoxine hydrochloride), vitamin B12 (cobalamin) and folic acid 1 mg (vitamin B8) are essential B vitamins that work as cofactors in reducing homocysteine levels in the blood stream. Homocysteine is an amino acid created in the digestion of protein. When proteins are metabolized, they are broken down into individual amino acids, including the sulfur-containing amino acid methionine. Methionine is broken down further to produce homocysteine, which once formed can be removed from the body in only two ways. It can be remade into methionine through a process called “remethylation”. This requires both folic acid and vitamin B12 where vitamin B12 functions as an essential “cofactor” in the reaction. Also, homocysteine can be made into the amino acid cysteine through a process called transulfuration, a process that requires two enzymes to work in concert with vitamin B6. Thus if someone ingests lots of protein, and there is not enough folic acid, vitamin B6, and vitamin B12 available to help digest it, homocysteine levels can build up in the bloodstream. Therefore, an increased level of these vitamins in the bloodstream result in a reduction of homocysteine levels. Studies have shown that oral folic acid supplements are effective in bringing down homocysteine levels. In a seesaw effect, as folic acid levels rise, levels of homocysteine drop.

The amounts are as follows: vitamin B6—2-50 mg, vitamin B12—25 mcg-2 mg; folic acid—0.01-5 mg.

In the preferred embodiment, the formulation is:
Policosanol—20 mg
DHA—400 mg
EPA—400 mg
vitamin B6 pyridoxine—25 mg
vitamin B12 cobalamin—400 mcg
folic acid—1 mg.

The ingredients are mixed together.

In addition to the above ingredients, the formulation can also contain healthy fillers such as gelatin, white beeswax, soy lecithin, glycerin, and carmel.

It is intended that the formulation can be administered orally to individuals, preferably before bedtime, and preferably on a daily basis in gel capsules, tablets form, or liquid form, or in any other pharmaceutically acceptable form, including food form.

The policosanol lowers LDL, raises HDL and thins the blood. DHA and EPA lowers triglycerides and to a certain extent thins the blood. The B vitamins reduce homocysteine levels.

Although the invention has been described primarily in connection with special and preferred embodiments, it will be understood that it is capable of modification without departing from the scope of the invention. The following claims are intended to cover all variations, uses, or adaptations of the invention, following, in general the principles thereof and including such departures from the present disclosure as come within known or customary practice in the field to which the invention pertains, or as are obvious to persons skilled in the field.

1. An oral formation for regulating total cholesterol and triglyceride levels and for reducing homocysteine levels in a human, comprising effective amounts of:
policosanol;
docosahexaenoic acid;
eicosapentaenoic acid;
vitamin B6;
vitamin B12; and
folic acid.

2. An oral formation for regulating total cholesterol and triglyceride levels and for reducing homocysteine levels in a human, comprising:
policosanol—2 mg-60 mg;
docosahexaenoic acid—20 mg-1000 mg;
eicosapentaenoic acid—20 mg-1000 mg;
vitamin B6—2 mg-50 mg;
vitamin B12—25 mcg-2 mg; and
folic acid—0.01 mg-5 mg.

3. An oral formation for regulating total cholesterol and triglyceride levels and for reducing homocysteine levels in a human, comprising:
policosanol—20 mg;
DHA—400 mg;
EPA—400 mg;
vitamin B6—25 mg;
vitamin B12—400 mcg; and
folic acid—1 mg.

4. A method of regulating total cholesterol and triglyceride levels and for reducing homocysteine levels, comprising periodically administering orally a formulation having effective amounts of:
policosanol;
docosahexaenoic acid;
eicosapentaenoic acid;
vitamin B6;
vitamin B12; and
folic acid.

5. A method of regulating total cholesterol and triglyceride levels and for reducing homocysteine levels, comprising periodically administering orally a formulation having:
policosanol—2 mg-60 mg;
docosahexaenoic acid—20 mg-1000 mg;
eicosapentaenoic acid—20 mg-1000 mg;
vitamin B6—2 mg-50 mg;
vitamin B12—25 mcg-2 mg; and
folic acid—0.01 mg-5 mg.

6. A method of regulating total cholesterol and triglyceride levels and for reducing homocysteine levels, comprising periodically administering orally a formulation having:
policosanol—20 mg;
DHA—400 mg;
EPA—400 mg;
vitamin B6—25 mg;
vitamin B12—400 mcg; and
folic acid—1 mg.
